(19) United States
(12) Patent Application Publication
(10) Pub. No.: US 2016/0029885 A1
(43) Pub. Date: Feb. 4, 2016

(54) IMAGE REFRACTION SYSTEM AND METHOD

(71) Applicant: SIRSVISION, LLC, Durham, NC (US)

(72) Inventors: Bruce J. HOOF, Durham, NC (US); Bruce M. SHIELDS, Burlington, NC (US)

(73) Assignee: SIRSVISION, LLC, Durham, NC (US)

(21) Appl. No.: 14/776,239

(22) PCT Filed: Oct. 16, 2013
(86) PCT No.: PCT/US13/65237

§ 371 Date: Sep. 14, 2015

Related U.S. Application Data
(60) Provisional application No. 61/791,831, filed on Mar. 15, 2013.

(51) Int. Cl. A61B 3/028; A61B 3/032; A61B 3/036

(52) U.S. Cl. CPC A61B 3/0285; A61B 3/036; A61B 3/032

(57) ABSTRACT

An image refractor, system and method are designed to improve refraction tests by allowing an examinee to compare, by viewing through at least one eye, at least two disparate images of the same acuity target, substantially side-by-side and simultaneously. The image refractor incorporates at least two prisms through which the acuity target is viewed. Embodiments may be attached to a standard phoropter and rotated into the visual axis so that the at least two images are produced by a combination of the lenses in the phoropter and the superimposed lenses of the image refractor. Embodiments determine the optimum correction for the sphere, cylinder, and axis of the refraction.

### CROSS-REFERENCE TO RELATED APPLICATIONS

This application is related to and claims priority to the filing date of Provisional Application Ser. No. 61/791,831 filed Mar. 15, 2013 of the same inventors herein, and entitled IMAGE DOUBLING REFRACTION SYSTEM AND METHOD. The disclosure of that Provisional Application is expressly incorporated herein by reference in its entirety.

### BACKGROUND

1. Field of the Invention

This invention relates to an image refraction system and method of refraction for simultaneously presenting at least two images of an acuity target to an examinee to aid in eye examination.

2. Discussion of Art

“Refraction” refers to the process of determining the appropriate power for a person's glasses or contact lenses to correct for myopia (nearsightedness), hyperopia (farsightedness), astigmatism (irregular image) and presbyopia (reading glasses). A refraction test is performed in the offices of ophthalmologists and optometrists and typically involves the use of an instrument called a “phoropter,” the basic version of which is conventional, well known and readily commercially available, for example, from companies such as Bausch and Lomb, Inc. A phoropter contains a series of lenses that are rotated into an examinee's visual axis until the optimum combination is found for that individual. Although an objective screening test may be performed first, with either an automated refractor or a procedure called “retinoscopy,” the fine tuning (and often the entire process) is achieved subjectively with the phoropter, in which the examinee is asked to compare two disparate images of the same acuity target (e.g., letters on a chart that are viewed through two differently refractive lenses) and determine which appears clearest to the examinee.

The decision as to what optical correction is needed to achieve the best vision is based on subjective responses to a series of sequentially presented differently refracted images. The optimum combination of lenses is determined based on an examinee's subjective responses to correct examinee's refractive error. It will be appreciated that the accuracy of determining the best corrective lenses is dependent on accuracy of information reported, which is less reliable when images are presented sequentially and memory is relied on.

With a conventional phoropter, the images are presented sequentially, with a slight difference in the lens power between the two. The examinee is expected to remember the appearance of the first image when comparing it to the second. Anyone who has been in the examinee's seat (just about everyone) knows that this is not easy. It often requires going back and forth several times between the two images, which is frustrating for the examinee, increases the testing time, and does not always insure the optimum answer.

According to current statistics from the American Academy of Ophthalmology, 32 million individuals in the United States have myopia, 12 million have hyperopia and 1 in 3 people have some degree of astigmatism. Virtually everyone over the age of 45 years has presbyopia and requires reading glasses. Approximately 150 million Americans wear glasses and spend $15 billion annually on eyewear. Publicly available data suggests there are 18,305 active ophthalmologists in the US (as of Dec. 31, 2009) and 36,000 optometrists. Worldwide, there are an estimated 200,000 ophthalmologists and 300,000 optometrists. Most ophthalmologists and optometrists in the US work out of two or more examination rooms, each of which is typically equipped with a phoropter. It is a conservative estimate, therefore, that there are over 100,000 conventional phoropters in use in the US and at least 500,000 worldwide.

There is a need to improve on the sometimes cumbersome process followed with current phoropter technology. Such improvements are provided by the invention described herein.

### BRIEF SUMMARY

Without in any way intending to limit the scope of the invention, which is defined by its claims, it can be said in general that the image refraction system of the invention improves the refraction aspect of eye examinations by allowing an examinee to compare two or more disparate images of a single acuity target, substantially simultaneously and, in many embodiments, substantially side-by-side. It is an improvement to and substantially different from current systems and methods that present two images sequentially and require the examinee to remember the appearance of each image in order to make a comparison.

In one specific aspect the invention relates to an image refraction system including a phoropter through which an examinee can view an acuity target, for example, a letter on a chart. An image refractor is associated with the phoropter. The image refractor includes at least two prisms, which produce two images of a single acuity target, with a refractive power (lens) on at least one side different from a refractive power on at least another side. The image refractor is mounted on the phoropter to be overlapped with a selected lens (or lenses) of the phoropter through which an examinee views the acuity target, to present two images of the target simultaneously and adjacent to each other, allowing the examinee to select the image which the examinee perceives as clearer. The combination of the phoropter and image refractor makes up the image refraction system in one exemplary embodiment.

In another aspect, the invention relates to a method of performing a refraction test on an examinee, involving placing an acuity target in front of one eye of the examinee. A lens comprised of a bi-prism having different refractive powers on respective sides is placed in front of at least one eye of the examinee to present two images, one of different resolution from the other, of the target. The examinee then indicates which image is clearer.

In still another aspect, the invention involves the image refractor as previously described, which is constructed for being moveably mounted on a phoropter to be used as also previously discussed. In other words, the image refraction system can either be permanently built into new phoropters, permanently attached to existing phoropters or temporarily attached to existing phoropters.

### BRIEF DESCRIPTION OF THE DRAWINGS

Having briefly described aspects of the invention, the same will become better understood from the following detailed description, made with reference to the accompanying drawings wherein:

FIG. 1 is a front view of a conventional prior art phoropter;

FIG. 2 is a front partial view of one embodiment of the image refraction system of the invention with an image refractor mounted on a phoropter, with a bi-prism shown constructed for use for one spherical lens correction of an examinee's vision attached to a conventional rotating arm of a phoropter;

FIG. 3 is a view as in FIG. 2 with the image refractor bi-prism rotated to be aligned with a lens (or lenses) of the phoropter to present two images side by side and simultaneously to an examinee;

FIG. 4 is a front view of one embodiment of a beam splitting bi-prism used in the system of FIGS. 2 and 3;

FIG. 5 is a side cross-section view of the bi-prism of FIG. 4, showing one side which is neutral refractive or plano, and the other side with a minus diopter refractive power selected for spherical correction;

FIG. 6 is a view as in FIG. 4 of another embodiment of a bi-prism employed with the invention incorporating two Jackson cross-cylinders for cylinder axis correction;

FIG. 7 is a view as in FIG. 5 showing the bi-prism with two Jackson cross-cylinders for cylindrical axis correction;

FIG. 8 is a view as in FIG. 4 of another embodiment of a bi-prism employed with the invention with a cylindrical lens on at least one side for cylindrical power correction;

FIG. 9 is a view as in FIG. 5 of a bi-prism with a cylindrical lens on at least one side used for cylindrical power correction;

FIG. 10 is a front view of an embodiment of an image refraction system showing an image refractor of the invention constructed with all three bi-prisms of FIGS. 4-9, and shown movably mounted on a phoropter; and

FIG. 11 is a view as in FIG. 10 with the bi-prism for spherical lens correction rotated into an examinee's visual axis.

### DETAILED DESCRIPTION OF EMBODIMENTS OF THE INVENTION

Embodiments of the invention are described hereafter, and depicted in the accompanying figures described previously. The figures and this description illustrate one or more embodiments, and, to the extent omitted, words referring to the described features as applying to “this embodiment” of the invention are to be implied throughout. As will be readily appreciated by those of ordinary skill in the art, these embodiments are not limiting.

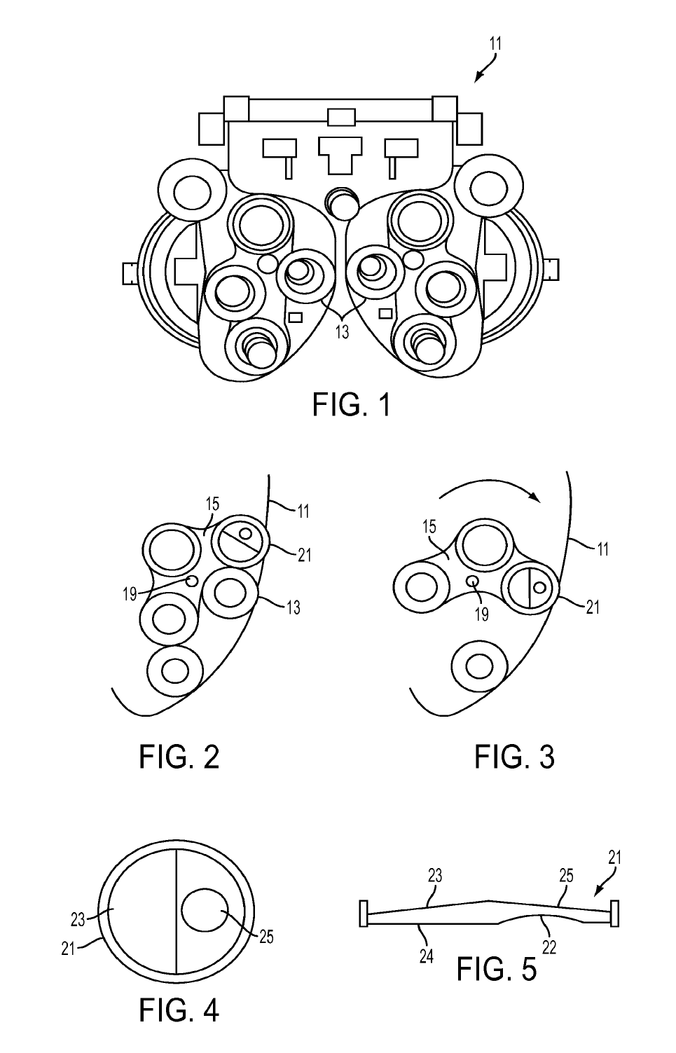

The system of the invention replaces the current technique of sequential presentation of two disparate images through a conventional prior art phoropter 11 shown in FIG. 1, by presenting the two images simultaneously. An examinee no longer has to remember the appearance of the previous image in order to determine which of the two is clearest but can compare the two in close proximity and substantially simultaneously. This is achieved in the described embodiments by an image refractor 15 and 51 as shown in FIGS. 2, 3, 10 and 11, which preferably attaches to the front (or back) of a standard phoropter 11 in a rotatable manner at axle 19 and 63. The image refractor 15 or 51 may be rotated into the visual axis of the phoropter so that the two images of the single test object (or visual acuity target) are produced by a combination of a lens 13 in the phoropter 11 and the superimposed lenses in the image refractor 15 or 51 of the invention. FIGS. 2 and 10 show the lens 13, and FIGS. 3 and 11 show the superimposed lens of the refractor 15 or 51, with lens 13 not shown because of the overlap. The image refractor, 15 and 51 is generally shown in FIGS. 2, 3, 10, and 11. The image refractors 15 and 51 incorporate bi-prisms, which in one embodiment split a single acuity target into two, side-by-side images presented simultaneously. In this case, a slight difference in refractive correction in the lens or lenses associated with one or both prisms allows the examinee to view two images of the same test object, presented to the examinee simultaneously and in close proximity, typically side-by-side, with a subtle difference between the two. When the bi-prisms are clear, the two images will appear against the same background without color variation, so that the difference in the two images is strictly due to a known amount of refractive correction on either side of the bi-prism. The examinee is then asked to determine which image is clearer, rather than having to remember the appearance of two images in sequential presentation.

As previously noted, the image refractor 15 and 51 is mounted on the phoropter at axle 19 and 63 to allow rotation of the different bi-prisms thereof in the field of vision of the phoropter 11.

To illustrate, with the current technology, an examinee may be asked to view a letter on the eye chart through a −1.00 diopter lens (for correcting mild myopia) in the phoropter, and then to view the same letter through a −1.25 or −1.50 diopter lens (with current sequential technology, it is often necessary to have a difference of at least 0.50 diopter or more in order for the examinee to distinguish a difference in the two images) and report which of the two images is clearer, based on their memory of the first image. With the image refraction system of the invention, the examinee may be asked to view the letter on the eye chart with a −1.00 diopter lens in the phoropter and plano (no additional refraction) on one side of the image refraction lens bi-prism and −0.25 on the other side, so that one of the two side-by-side images is seen through a −1.00 lens and the other through a −1.25 lens (i.e., a combination of lenses in phoropter and the bi-prism lens) and to report which of the two images is clearer. This is shown, for example, in FIGS. 2, 3, 4, and 5 with prism lens 21 having a prism surface 23 and prism surface 25 making up the bi-prism 21, for example, as a minus 0.25 diopter lens superimposed with the prism. The prism lens 21 also includes plano surface 24 and concave refractive surface 22. By viewing the two images presented simultaneously side-by-side, it is anticipated that less refractive difference in the two images will be needed for the examinee to appreciate a difference, adding to the precision of the process, although the invention embodies any combination of refractive lenses on either side of the bi-prism.

In the case of the bi-prisms 21, 27 and 33 shown in FIGS. 5, 7, and 9, it will be appreciated that the top surfaces generally shown as 23, 25, 26, 28, 34 and 38 are prism surfaces which as paired make up bi-prism lenses 21, 27 and 33. Thus, in FIG. 5, bi-prism lens 21 includes prism surfaces 23 and 25 and refractive surfaces 24 (plano) and 22 (concave). In FIG. 7, the bi-prism lens 27 includes adjacent prism surfaces 26 and 28, and refractive surfaces 31 and 32. In FIG. 9, the bi-prism lens 33 includes adjacent prism surfaces 34 and 38, and refractive surfaces 36 and 37. The prism surfaces and refractive surfaces may be manufactured in a single grinding process, or assembled from multiple optical components which can be, in one embodiment, for example, cemented or mechanically held together.

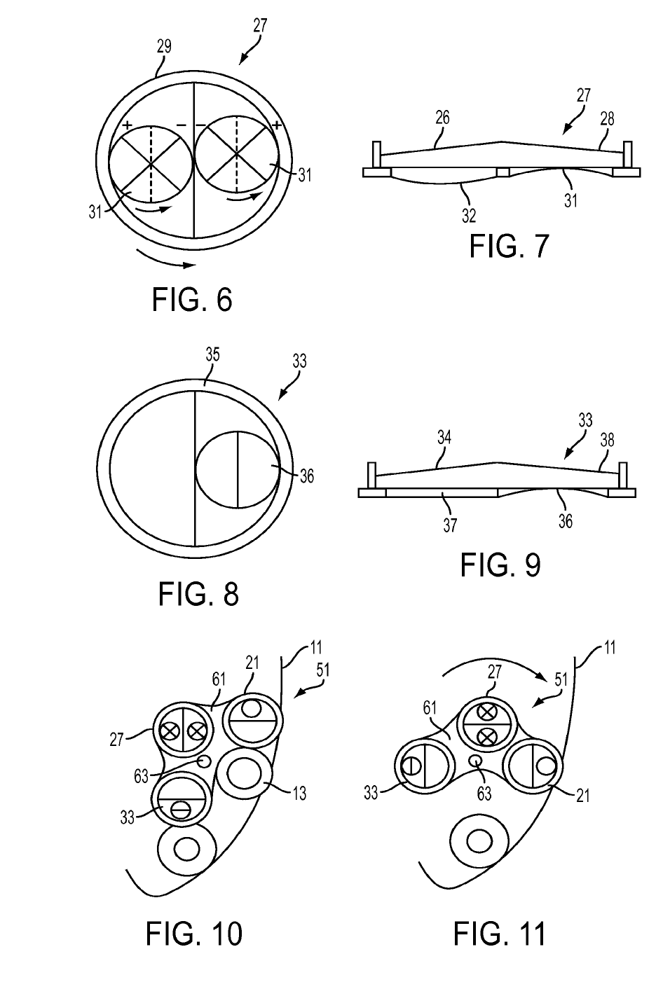

In one embodiment as shown in FIGS. 10, and 11, the image refraction system may be made up of three separate bi-prism lenses 21, 27, and 33. Bi-prism lens 21 as shown in FIGS. 2-5, 10, and 11 is for spherical correction (which corrects for myopia, hyperopia and presbyopia). Bi-prism lens 33 shown in FIGS. 8-11 is for cylindrical correction 36. Bi-prism lens 27 shown in FIGS. 6, 7, 10, and 11 is for cylindrical axis correction 31. Bi-prism lenses 33 and 27 correct for astigmatism. All three devices in this embodiment incorporate lenses, including on at least one surface of the bi-prisms which split an acuity target into two, side-by-side images presented simultaneously. The bi-prism is made up of two prisms with powers, expressed in prism diopters, selected to produce an optimum separation between two images. The bi-prisms may be mounted in a stable frame or can be designed to rotate within the frame so that either side of the bi-prisms can be presented to the examinee's right or left eye (the latter is occasionally useful to ensure that the examinee is giving reliable responses).

In one embodiment of a device shown in FIGS. 4, 5, 10, 11 including a spherical correction prism lens 21, one refractive surface 24 of the bi-prism lens has no refractive power (piano) or a built-in correction of +0.25 diopter or more, while the other refractive surface 22 may have a built-in correction of −0.25 diopter or more. As each conventional spherical lens 13 of the phoropter is presented, the examinee sees two images of the acuity target simultaneously with the difference between them being the difference in spherical power on the two sides of the bi-prism. Depending on the examinee's preference between the two images (i.e., which is clearer), the spherical lenses 13 in the phoropter are further changed to interact with the spherical lens (or lenses) in the bi-prism, until an end point is reached. For example, with a lens (not shown) in the phoropter, the examinee may prefer an image caused by the plano refractor 24 of the bi-prism 21, while, with another lens (not shown) of the phoropter, they may prefer the image caused by −0.25 concave refractive surface 22. At this point, the image refractor 15 or 51 is withdrawn from the visual axis and the examinee may be allowed to choose between the two phoropter lenses (or a duochrome test may be used for the final determination).

In an embodiment of the device for cylindrical correction shown in FIGS. 8-11, one refractor surface 37 of the bi-prism lens 33 has no refractive power or a cylindrical correction power of +0.25 diopter or more. The other refractive surface 36 may have a cylindrical correction power of −0.25 diopter or more. The lens(es) system can rotate freely within a fixed setting of the frame to align with the axis of the cylinder in the phoropter. The bi-prism/lens system 33 is surrounded by a knurled ring 35 that is in contact with the cylindrical correction lens(es) so that rotation of the ring 35 for the bi-prism lens of FIG. 7 causes the lens 36 to rotate. The axis of the cylindrical lens(es) in the device is always aligned with the axis of the cylindrical lens 13 in the phoropter 11, either by manual rotation (i.e., with the examiner's fingers) of the knurled ring 35 for the bi-prism of FIG. 6 through direct, mechanical connection with the phoropter 11, (i.e., rotating the cylindrical lens in the phoropter automatically rotates the cylindrical lens in the image refraction system by the same amount and in the same direction) or electronically in remotely controlled phoropters. The mechanical linkage with the phoropter can be made in a mechanical connection which will be well known to those of ordinary skill, or an electronic control arrangement could be utilized. In the former instance, when the axis of a cylindrical lens in the phoropter is manually rotated in a standard manner, the axis of the cylindrical lens in the bi-prism 33 automatically rotates the same amount. As with the spherical correction, the cylindrical lenses in the phoropter are changed according to the examinee's preference until the end point is reached, at which point the device is withdrawn from the visual axis and the final determination is made.

In an embodiment of a device for correcting the axis of the cylinder as shown in FIGS. 6, 7, 10, and 11, two Jackson cross-cylinders 31 are positioned behind or in front of the bi-prisms in a fixed setting that allows them to rotate freely. The Jackson cross-cylinder is a conventional lens device used to determine the proper axis of the cylindrical lens to correct for astigmatism. As with the lens for cylindrical correction, a knurled ring 29 surrounds the bi-prism 27 and is in contact with the two cross-cylinders in such a way that they rotate in the same direction when the ring 29 is rotated. The powers of the two cross-cylinders 31 are reversed so that the axis of the minus cylinder behind or in front of one prism is at 90° the axis of the cylindrical lens behind or in front of the other prism. The axes of the cross-cylinders 31 are always aligned with the axis of a lens in the phoropter either by manual rotation of the knurled ring 29 through direct, mechanical connection with the phoropter, or electronically in remotely controlled phoropters, as previously described for determining cylindrical power. In the mechanical connection instance, when the cylinder axis in the phoropter is rotated in the standard manner, the axes of the cross-cylinders 31 automatically rotate the same amount. As with the other two devices, the axis of the cylindrical lens in the phoropter is rotated in accordance with the examinee's preference of cross-cylinders in the two images until an end point is reached, at which point the device is withdrawn from the visual axis.

In the embodiment of FIGS. 10 and 11, the three specific devices or lenses 21, 27, and 33 (for correction of sphere, cylinder, and cylinder axis) are shown all mounted on a single arm 61 with an axle 63 that attaches to the phoropter 11 and allows the arm to rotate in such a way that any one of the three devices 21, 27, and 33 can be rotated into the visual axis of the phoropter. In one embodiment, the complete system can be mounted on the front of a standard phoropter 11, replacing the standard rotating arm that contains the Jackson cross-cylinder and a prism, or with a special mounting on the back of the phoropter 11, closer to the examinee's eye. In an alternate embodiment, the system can be independent of the mechanism of the phoropter 11, with all movements of the arm and lenses in the bi-prisms accomplished manually, or it can be linked to the phoropter 11 in such a way that movement of the cylindrical axis in the phoropter 11 automatically rotates the axes in the devices. In still another embodiment, it can also be incorporated into an electronic phoropter, so that all functions of the system are achieved by remote control. FIGS. 2 and 3 show a system similar to FIGS. 10 and 11 with three lens prisms, but two of them (i.e., the conventional Jackson cross-cylinder and a prism) are not characterized herein. As may be appreciated, one embodiment may delete two of the lens prisms leaving only the lens prism 21.

Another embodiment of the image refraction system is to have a single bi-prism (rather than one for each of the three lens systems), which is either mounted permanently in the visual axis of the phoropter so the examinee is always seeing the double image or can be clipped or rotated into the visual axis when desired. The three lenses on the rotating arm would then have only the refractive corrections as described in the preceding paragraph.

Yet another embodiment variation of the image refraction system is to use the bi-prism for spherical correction alone, which can be attached to the existing rotating arm on the standard phoropter or clipped directly over the visual axis of the phoropter. This device would be limited to determining the optimum spherical correction but would have the advantage of simplicity to manufacture (no moving parts) and significantly lower production and sales costs. The image refraction system, therefore, has a wide range of production options, from the simple spherical correction to the complete set of three bi-prisms on a dedicated rotation arm, which can either be attached to an existing phoropter or incorporated into new phoropters with either manual operation or electronic, remote control.

It is believed that eye examinations utilizing various embodiments of the image refraction system will be favored over the current technique of refraction by both examinees and eye care providers for any or all of the following reasons. Examinees will find it easier to perform as they will no longer have to remember what an image looked like in order to compare it with another image, and can compare images side-by-side. Providers will appreciate the anticipated reduced time requirement (instead of having to go back and forth between two images, often many times, the images are presented together only one time). All will benefit from the anticipated enhanced accuracy in finding the examinee's best correction. As a result, it is believed that instrument companies will want to incorporate this new technology into their phoropters (to stay ahead of their competition), whether incorporated within the structure of a phoropter or as an attachment thereto, and ophthalmologists, optometrists and eye care service providers will want to have the image refraction system in their offices (to provide the best care for their patients).

### EXAMPLES

In an experiment by one of the inventors using a standard loose lens bi-prism (two six-prism diopter, base-in prisms) and a standard phoropter, the experiment provided proof of concept that two, side-by-side images of a letter on a standard eye chart can both be seen clearly and equally when the bi-prisms used in the invention are placed either in front of or behind the visual axis of the phoropter. In addition, when a second loose lens, e.g., −0.25 for sphere correction, is held just in front of one side of the bi-prism, the two images are still visible, with a slight difference in the clarity of the two, resulting from the difference in refractive correction on either side of the bi-prism.

While illustrative examples show the use of bi-prisms to present two adjacent images simultaneously, the prism could be constructed to present three or more images to allow even more precise selection resulting in better refractive correction through prescription of more precise correcting eyeglass lenses.

Other implementations may involve any number of different images or targets, including different letters in capital or lower case, symbols, pictures, geometric designs, or letters from other languages. Images of different sizes could be presented or different images, such as the letter A and a square, could be presented for comparison.

Each of the foregoing examples and embodiments involves the simultaneous presentation of differently refracted images, i.e., at least two, of the same acuity target, which are differently refracted images of said target. Such presentation may be on a common background, e.g., single color, or different colors as discussed hereafter. The simultaneous presentations proceed until based on information from the examinee from each simultaneous comparison, the optimum combination of lenses is identified. This technique and system allows measurement of eyesight more accurately allowing refraction correction never previously possible. The examinee is no longer required to rely on memory because the images are presented simultaneously, eliminating errors based on faulty memory.

The images could be presented in specific orientations relative to each other. Similarly, they could be presented in different colors or on different color backgrounds, which may be helpful for discrimination in the case of more than two images being presented. This can be achieved through colored prisms or lenses, optical filters, detachable filters, or even coatings.

The side by side presentation allows an examinee to distinguish between images having a lesser difference in refraction than with sequential presentation. For example, while values of ±0.25 diopter have been discussed, they are only illustrative. It is anticipated that prisms with values of less than ±0.25 may be used with improved manufacturing techniques, for example, at values of ±0.1.

Thus, with the invention a degree of refraction between two images can be presented which is better than that with conventional technologies. In performing a refraction with a conventional phoropter a difference of 0.25-0.5 diopters is typically used. With the invention, one of the simultaneous images is viewed through, for example, the phoropter's −1.00 lens and a second image may be presented through a combination of the phoropter lens and a −0.25 diopter lens of the invention resulting in a diopter value of −1.25. Moreover, examinees should prefer the use of the invention because simultaneous presentation makes selection easier. The amount of time for examinations to be conducted should also be reduced.

In yet still another embodiment, a method of performing a refraction test on an examinee includes placing a single acuity target in front of one eye of an examinee. At least two images are simultaneously virtually presented, one being presented to be perceived by the examinee as being differently refracted from another. A response is then received from the examinee indicating which of the virtually presented at least two images is preferred.

While several optical arrangements have been described herein, it will be appreciated by those of ordinary skill that a number of different and alternative optical arrangements can be implemented to achieve the method described in the preceding paragraph.

The foregoing details are exemplary only. Other modifications that might be contemplated by those of ordinary skill in the art are within the scope of this invention, and are not limited by the examples illustrated herein.

What is claimed is:

1. A refraction system, comprising:
a phoropter having plural lenses through which an examinee can view an acuity target presented to the examinee through a selected one of said plural lenses, said plural lenses having optical powers differing from each other; and
an image refractor comprising a bi-prism with a refractive lens power on at least one side thereof differing from a refractive power on at least another side thereof, said image refractor mounted on the phoropter in a manner allowing said image refractor in at least one position to be overlapped with said selected lens for presenting said acuity target to the examinee as at least two images of adjacent targets, each differently refracted from the other target.

2. The refraction system of claim 1, wherein said at least two prisms are constructed for presenting said at least two adjacent images of a visual acuity target images in a horizontal side by side view.

3. The refraction system of claim 1, wherein said at least two prisms are constructed for presenting said at least two adjacent target images in a vertical side by side view.

4. The refraction system of claim 1, wherein said image refractor is constructed with more than two prisms constructed for presenting more than two acuity target images differently refracted one from the other, whereby the examinee can select an acuity target image the examinee perceives as clearer than others.

5. The refraction system of claim 1, wherein said image refractor is constructed for allowing viewing of differently refracted images simultaneously.

6. The refraction system of claim 1, wherein said image refractor is constructed for presenting the images on a colored background.

7. The refraction system of claim 6, wherein the image refractor is constructed so that the target images are presented as colored images in differing colors from each other.

8. The refraction system of claim 1, wherein said image refractor prism has a neutral diopter refractive power on one side and a refractive power on the other side of about ±0.25 diopter.

9. The refraction system of claim 1, wherein said image refractor prism has a diopter refractive power on one side of about +0.25 diopter and a refractive power on the other side of about −0.25 diopter.

10. The refraction system of claim 1, wherein said image refractor prism is comprised of a bi-prism and said image refractor comprises at least three bi-prisms arranged on a phoropter in an arrangement for being rotated into alignment with a selected lens of said phoropter which is positioned for viewing a target.

11. The refraction system of claim 10, wherein said at least three bi-prisms are constructed respectively to correct for eye sphere, cylinder, and axis.

12. The refraction system of claim 11, wherein said at least three bi-prisms are mounted in a revolving arrangement on the front of the phoropter in a manner for each bi-prism is placeable in alignment with a viewing lens of the phoropter.

13. A method of performing a refraction test on an examinee, comprising:
placing an acuity target in front of at least one eye of the examinee;
placing in front of at least one eye of the examinee at least one lens comprised of at least one bi-prism, respective sides of the bi-prism having a different refractive power than the other side thereby presenting two differently refracted images of the target to the examinee; and
determining from the examinee which of the two images of the target appear clearer to the examinee.

14. The method of claim 13, further comprising said bi-prism being shaped to have the refractive powers of the respective sides at a value for determining the sphere of the eye.

15. The method of claim 13, further comprising said bi-prism being shaped to have the refractive powers of the respective sides at a value for determining the cylinder of the eye.

16. The method of claim 13, further comprising said bi-prism being shaped to have the refractive powers of the respective sides at a value for determining the axis of the eye.

17. A device for use in an image refraction system, comprising:
an image refractor comprised of at least one bi-prism with a refractive power on at least one side thereof differing from the refractive power of at least another side thereof; and
said image refractor having a mounting structure, said mounting structure constructed to retain the image refractor in a movable relationship relative to a lens of a phoropter such that an examinee viewing an acuity target will view the acuity target through the image refractor, said view substantially simultaneously presenting to the examinee at least two adjacent images of the acuity, one refracted differently from another.

18. The device of claim 17, wherein said at least one bi-prism is constructed for presenting said at least two adjacent target images in a horizontal side by side view.

19. The device of claim 17, wherein said at least one bi-prism is constructed for presenting said at least two adjacent target images in a vertical side by side view.

20. The device of claim 17, wherein said image refractor is constructed with the bi-prism replaced with a plural prism constructed for presenting more than two acuity target images differently refracted one from the other, whereby the examinee can select an acuity target the examinee perceives as clearer than others.

21. A method of performing a refraction test on an examinee, comprising:
placing in front of one eye of an examinee at least two images of a single acuity target, said images displayed to be viewed simultaneously by said eye, at least one of said at least two images presented to be perceived by the examinee as being differently refracted from another of said at least two images; and
receiving from the examinee a response indicating which of the at least two images is preferred.
